(19) United States
(12) Patent Application Publication
(10) Pub. No.: US 2022/0141600 A1
(43) Pub. Date: May 5, 2022

(54) HEARING ASSISTANCE DEVICE AND METHOD OF ADJUSTING AN OUTPUT SOUND OF THE HEARING ASSISTANCE DEVICE

(71) Applicant: PixArt Imaging Inc., Hsin-Chu County (TW)

(72) Inventors: Cheng-Te WANG, Taipei (TW); Jian-Ying LI, Taipei (TW); Kuo-Ping YANG, Taipei (TW)

(21) Appl. No.: 17/241,132

(22) Filed: Apr. 27, 2021

(30) Foreign Application Priority Data
Nov. 2, 2020 (CN) 202011205472.2

(51) Int. Cl. H04R 25/00; G10L 21/034

(52) U.S. Cl. CPC H04R 25/505; H04R 2430/01; G10L 21/034

(57) ABSTRACT

A hearing assistance device and method of adjusting an output sound of the hearing assistance device are disclosed. The method comprises the following steps: emitting a test sound, wherein the frequency of the test sound is higher than 15 kHz and lower than 30 kHz; receiving a response sound after the test sound is emitted; determining whether the response sound is higher than a response sound threshold; and, if yes, adjusting an output volume.

### BACKGROUND OF THE INVENTION

### 1. Field of the Invention

The present invention relates to a hearing assistance device and a method of adjusting an output sound of the hearing assistance device; more particularly, the present invention relates to a hearing assistance device and a method of adjusting an output sound of the hearing assistance device capable of adapting to deformation of the shape of an ear canal caused by human facial movements, for example mandibular movements like speaking, chewing or swallowing, by emitting a high frequency test sound into a user's ear canal, detecting ear an canal frequency response of the high frequency test sound within the user's ear canal; and determining a user behavior mode such that the volume of the sound from the user wearing the hearing assistance device can be adjusted.

### 2. Description of the Related Art

Generally, most hearing-impaired people or people in need of hearing assistance wear a hearing assistance device (such as a hearing aid or a headset equipped with a hearing assistance function) to clearly hear external sounds. However, most conventional hearing assistance devices cannot distinguish whether the sounds come from external environment or from the user wearing the hearing assistance device (such as the sounds of the user's own voice, or the sounds of chewing or swallowing). Because the hearing assistance device will unconditionally amplify all received sounds, the sounds of the user's own voice will be amplified by the hearing assistance device as well, such that the user who wears the hearing assistance device may experience discomfort caused by hearing excessively loud sounds of the user's own speech.

Conventionally, it is known that both an inner microphone and an outer microphone can be installed in a hearing assistance device in order to detect the user's own voice or environmental sounds. For example, U.S. Patent Application Publication No. 2004/0202333A1, U.S. Pat. No. 9,369,814B2, U.S. Pat. No. 10,171,922B2 and European Patent Application Publication No. 1640972A1 have disclosed such technologies. U.S. Patent Application Publication No. 2004/0202333A1 utilizes energy level differences or frequency differences between sound signals received by the inner microphone and the outer microphone to determine whether the hearing assistance device is malfunctioning U.S. Pat. No. 10,171,922B2 and European Patent Application Publication No. 1640972A1 utilize energy level differences, time differences or frequency differences between sound signals received by the inner microphone and the outer microphone to determine whether the received sound is the user's own voice or an environmental sound.

In addition, a facial movement detector can be used to determine whether the user is speaking. For example, U.S. Pat. No. 9,225,306 discloses a hearing assistance device with a built-in facial movement detector for determining and helping to adjust the voice of a user wearing the hearing assistance device such that the voice from the user wearing the hearing assistance device will not be amplified too greatly. U.S. Pat. No. 10,021,494 discloses a hearing assistance device with a built-in vibration-sensitive transducer for determining whether the detected vibration requires further sound processing so as to achieve the effects of power saving and improving user comfort.

As a result, in the technology development of hearing assistance devices, it is a very important task to determine whether sounds received by the hearing assistance device are sounds that need to be amplified or sounds of the user's own voice. Recent publications such as “Acoustic Ear Recognition for Person Identification” and “Analysis of Deformation of the Human Ear and Canal Caused by Mandibular Movement” have discussed the deformation of the shape of the human ear canal during speaking, chewing and swallowing. Therefore, it is possible to detect the determination of the user's ear canal to determine the user's current behavior mode and thereby to determine whether a sound is the user's own voice or a sound that needs to be amplified. However, such application has not been implemented in the field of hearing assistance devices yet, so there is still room fir developing and improving the user comfort of hearing assistance devices.

Therefore, there is a need to provide a hearing assistance device and a method of adjusting the output sound of the hearing assistance device to mitigate and/or obviate the aforementioned problems.

### SUMMARY OF THE INVENTION

It is an object of the present invention to provide a hearing assistance device capable of adapting to deformation of the shape of an ear canal caused by human facial movements by emitting a high frequency test sound into a user's ear canal, detecting an ear canal frequency response of the high frequency test sound within the user's ear canal, determining a user behavior mode, and then adjusting the volume of the sound from the user wearing the hearing assistance device.

It is another object of the present invention to provide a method capable of adapting to determination of the shape of an ear canal caused by human facial movements by emitting a high frequency test sound into a user's ear canal, detecting an ear canal frequency response of the high frequency test sound within the user's ear canal, determining a user behavior mode, and then adjusting the volume of the sound from the user wearing the hearing assistance device.

To achieve the abovementioned objects, the hearing assistance device of the present invention comprises a speaker, an in-ear speaker, an in-ear sound receiver and a sound processing unit. The in-ear speaker is used for emitting a high frequency test sound, wherein the frequency of the high frequency test sound is higher than 15 kHz and lower than 30 kHz. The in-ear sound receiver is used for receiving a response sound after the in-ear speaker emits the high frequency test sound. The sound processing unit is used for determining whether the response sound is higher than a response sound threshold, and, if yes, adjusting an output volume of the speaker.

The present invention further provides a method of adjusting an output sound of a hearing assistance device which is applicable for being used in a hearing assistance device. The method of adjusting an output sound of a hearing assistance device comprises the following steps: emitting a high frequency test sound, wherein the frequency of the high frequency test sound is higher than 15 kHz and lower than 30 kHz; receiving a response sound after the high frequency test sound is emitted; determining whether the response sound is higher than a response sound threshold; and, if yes, adjusting an output volume.

The hearing assistance device and the method of adjusting an output sound of a hearing assistance device of the present invention are capable of adapting to deformation of an ear canal caused by human facial movements for example mandibular movements like speaking, chewing or swallowing, by emitting a high frequency test sound into a user's ear canal, detecting an ear canal frequency response of the high frequency test sound within the user's ear canal, determining a user behavior mode, and thereby identifying whether the sound received by the hearing assistance device is the voice of the user wearing the hearing assistance device. If yes, the hearing assistance device will lower the volume; otherwise, the hearing assistance device will not adjust the volume. As a result, the present invention can achieve the object of making the hearing assistance device adjust the volume of the user's own voice and thereby improving the deficiency of existing techniques.

Other objects, advantages, and novel features of the invention become more apparent from the follow detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only and not as a definition of the invention.

In the drawings, wherein similar reference numerals denote similar elements throughout the several views:

FIG. 1 illustrates a device structural drawing of a hearing assistance device of the present invention.

FIG. 2 illustrates a flowchart of a method of adjusting an Output sound of the hearing assistance device in a first embodiment of the present invention.

FIG. 3 illustrates a flowchart of the method of adjusting an output sound of the hearing assistance device in a second embodiment of the present invention.

### DETAILED DESCRIPTION OF THE PREFERRED EMBODIMENT

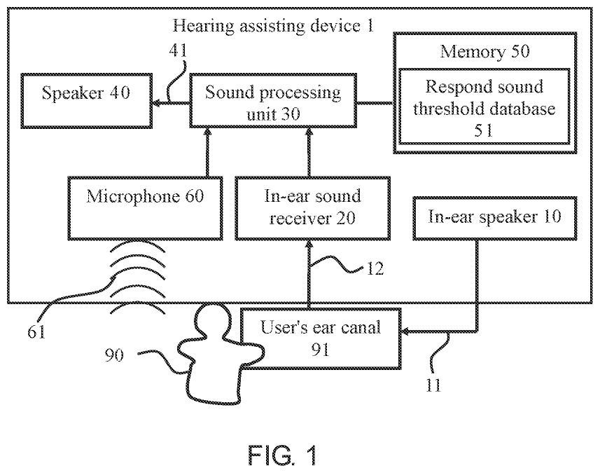

Please refer to FIG. 1, which illustrates a device structural drawing of the hearing assistance device of the present invention.

As shown in FIG. 1, in one embodiment of the present invention, the hearing assistance device 1 of the present invention comprises an in-ear speaker 10, an in-ear sound receiver 20, a sound processing unit 30, a speaker 40, a memory 50 and a microphone 60, wherein the in-ear sound receiver 20, the speaker 40, the memory 50 and the microphone 60 are electrically connected to the sound processing unit 30. Generally speaking, the sound processing unit 30 is mainly used for executing functions of the hearing assistance device 1 after the microphone 60 receives a speech signal 61, such as functions of frequency shift and frequency variation. If this invention is applied in a digital hearing assistance device, the function may include conversion between analog signals and digital signals.

As shown FIG. 1, in one embodiment of the present invention, the in-ear speaker 10 is used for emitting a high frequency test sound 11 to a user's ear canal 91. The in-ear sound receiver 20 is used for receiving a response sound 12 generated after the high frequency test sound 11 is reflected by the user's ear canal 91. The sound processing unit 30 is used for determining whether the response sound 12 is higher than a response sound threshold pre-stored in a response sound threshold database 51 of the memory 50. If yes, it means that the currently received speech signal 61 comes from the user 90 (such as the sounds of the user's own voice, or the sounds of chewing or swallowing). At this time, the sound processing unit 30 will lower an output volume 41 outputted from the speaker 40 so as to prevent the hearing assistance device 1 of the present invention from unconditionally amplifying the user 90's own voice. If the sound processing unit 30 determines that the response sound 12 is lower than the response sound threshold, it means that the currently received speech signal 61 does not come from the user 90. At this time, the sound processing unit 30 will not adjust the output volume 41 outputted from the speaker 40. Please note that in one embodiment of the present invention, the in-ear speaker 10 can be integrated with the speaker 40 so that it may emit the high frequency test sound 11 when the speaker 40 is not in use, or it may mix the high frequency test sound 11 with the output volume 41.

According to one embodiment of the present invention, each of the different types of user behavior modes is respectively defined with a corresponding response sound threshold. The user behavior modes include, for example, the user 90 speaking, chewing or swallowing. The sound processing unit 30 will determine whether the response sound 12 is higher than a response sound threshold corresponding to a user behavior mode pre-stored in the response sound threshold database 51. If yes, it means that the currently received speech signal 61 comes from the user 90 (such as the sounds of the user's own voice, or the sounds of chewing or swallowing). At this time, the sound processing unit 30 will lower an output volume 41 outputted from the speaker 40. For example, if the response sound 12 received by the in-ear sound receiver 20 is higher than the response sound threshold corresponding to the user 90's chewing behavior pre-stored in the response sound threshold database 51, it is then determined that the currently received speech signal 61 is the sound of the user 90 chewing food, and the sound processing unit 30 will lower the output volume 41 outputted from the speaker 40.

If the sound processing unit 30 determines that the response sound 12 is lower than the response sound threshold corresponding to the user behavior mode, it means that the currently received speech 61 does not come from the user 90. At this time, the sound processing unit 30 will not adjust the output volume outputted from the speaker 40 so that the user 90 can hear the speech signal 61 clearly. According to one embodiment of the present invention, the mechanism of the sound processing unit 30 determining whether the response sound 12 is lower than the response sound threshold corresponding to the user behavior mode can be achieved by means of comparing the volume of the response sound within a specific frequency band between 15 kHz and 30 kHz, wherein the specific frequency band is subject to the frequency of the high frequency test sound 11.

Please note that, to avoid interference with the hearing of the user 90, the frequency of the high frequency test sound 11 sent from the in-ear speaker 10 is higher than 15 kHz and lower than 30 kHz. According to one preferred embodiment of the present invention, the frequency of the high frequency test sound 11 is higher than 16 kHz and lower than 20 kHz. Please note that, according to one embodiment of the present invention, the response sound threshold database 51 can store a plurality of response sound thresholds which each respectively correspond to a user behavior mode. For example, the user behavior modes such as the user's own speaking, chewing and swallowing respectively have corresponding response sound thresholds. Therefore, whenever the sound processing unit 30 determines that the response sound 12 is higher than the response sound threshold of any user behavior mode, the sound processing unit 30 will then lower the output volume outputted from the speaker 40.

Please note that the shape of each individual's ear canal will be correspondingly deformed according to different individual behaviors such as eating and speaking, and the frequency responses of the high frequency test sound 11 reflected within ear canals of different shapes are subject to different behavior modes of different persons; therefore, it is required that the user 90 establish a corresponding response sound threshold database 51 prior to the first use of the hearing assistance device 1 of the present invention. In this embodiment, when the user 90 follows instructions from the hearing assistance device 1 to take different actions, the in-ear speaker 10 will play a test audio signal within a frequency range, and the in-ear sound receiver 20 will receive sounds accordingly, in order to analyze the frequency response dynamic modes of the user's ear canal 91 in different behavior states (such as speaking, chewing or swallowing), and to utilize the data for calculating the response sound thresholds each respectively corresponding to one of the different user behavior triodes, so that the response sound thresholds can be later used as a comparison reference specifically for the user 90 wearing the hearing assistance device 1.

Please note that each of the above modules not only can be configured as a hardware device, a software program, firmware or a combination thereof but also can be implemented as a circuit loop or other equivalent and appropriate forms; further, each of the modules can be configured either independently or jointly. Moreover, the abovementioned embodiments only describe preferred embodiments of the present invention. To avoid redundant description, not all possible variations and combinations are described in detail in this specification. However, those skilled in the art will understand that the above modules or components are not all necessary parts. In order to implement the present invention, other more detailed known modules or components might also be included. It is possible that each module or component can be omitted or modified depending on different requirements, and it is also possible that other modules or components might be disposed between any two modules.

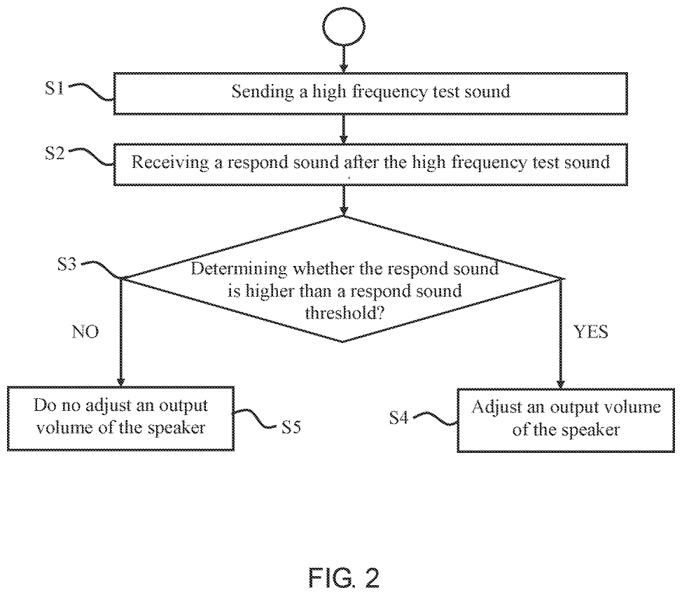

Next, please refer to both FIG. 1 and FIG. 2. FIG. 2 illustrates a flowchart of a method of adjusting an output sound of the hearing assistance device according to a first embodiment of the present invention. The components provided in FIG. 1 will be referenced for sequentially describing steps S1 to S5 as shown in FIG. 2.

Step S1: emitting a high frequency test sound.

The in-ear speaker 10 of the hearing assistance device 1 is used for emitting a high frequency test sound 11 into the user's ear canal 91. Please note that to avoid interference with the user's hearing comfort, the frequency of the high frequency test sound 11 sent from the in-ear speaker 11 is higher than 15 kHz and lower than 30 kHz. According to one preferred embodiment of the present invention, the frequency of the high frequency test sound 11 is higher than 16 kHz and lower than 20 kHz.

Step S2: receiving a response sound after the high frequency test sound is emitted.

The in-ear sound receiver 20 of the hearing assistance device 1 is used for receiving a response sound 12 generated after the high frequency test sound. 11 is reflected by the user's ear canal 91.

Step S3: determining whether the response sound is higher than a response sound threshold.

The sound processing unit 30 of the hearing assistance device 1 is used for determining whether the response sound 12 is higher than a response sound threshold pre-stored in a response sound threshold database 51 of the memory 50. If yes, it means that the currently received speech signal 61 comes from the user 90 (such as the sounds of the user's own voice, or the sounds of chewing or swallowing). At this time, the sound processing unit 30 will lower the output volume 41 outputted from the speaker 40 (step S4) so as to prevent the hearing assistance device 1 of the present invention from unconditionally amplifying the user 90's own voice. If the sound processing unit 30 of the hearing assistance device 1 determines that the response sound 12 is lower than the response sound threshold corresponding to the user behavior mode, it means that the sound does not come from the user 90. At this time, the sound processing unit 30 will not adjust the output volume outputted from the speaker 40 (step S5) so that the user 90 can hear the speech signal 61 clearly.

According to one embodiment of the present invention, the mechanism of the sound processing unit 30 determining whether the response sound 12 is lower than the response sound threshold corresponding to the user behavior mode can be achieved by means of comparing the volume of the response sound within a specific frequency band between 15 kHz and 30 kHz, wherein the specific frequency band is subject to the frequency of the high frequency test sound 11.

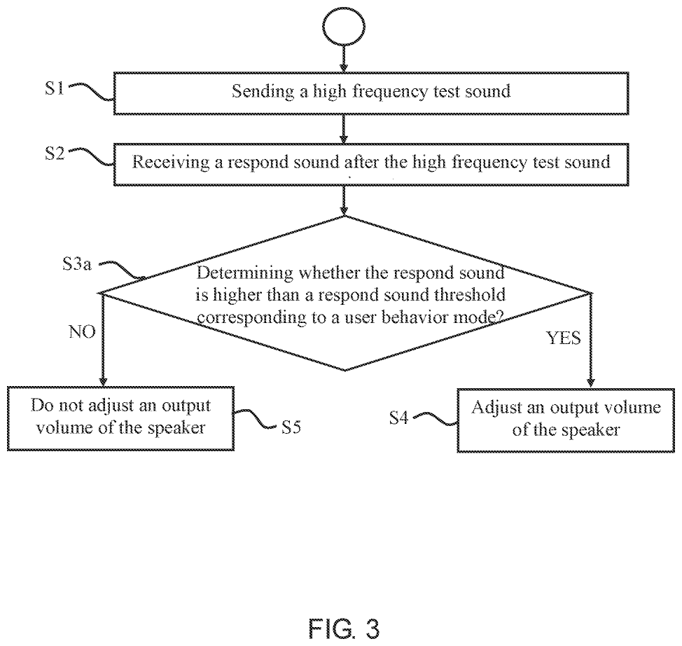

Please refer to both FIG. 1 and FIG. 3. FIG. 3 illustrates a flowchart of the method of adjusting an output sound of the hearing assistance device in a second embodiment of the present invention. In the second embodiment, the method of the present invention comprises steps S1, S2, S3a, S4 and S5, wherein steps S1, S2, S4 and S5 are the same as the steps disclosed in the first embodiment and require no further description; therefore, only the details of step S3a are described hereinafter.

Step S3a: determining whether the response sound is higher than a response sound threshold corresponding to a user behavior mode.

In the present invention, each of the different types of user behavior modes is respectively defined with a corresponding response sound threshold. The user behavior modes include, for example, the user 90 speaking, chewing or swallowing. The sound processing unit 30 will deter mine whether the response sound 12 is higher than a response sound threshold corresponding to a user behavior mode pre-stored in the response sound threshold database 51. If yes, it means that the currently received speech signal 61 comes from the user 90 (such as the sounds of the user's own voice, or the sounds of chewing or swallowing). At this time, the sound processing unit 30 will lower an output volume 41 outputted from the speaker 40 (step S4). For example, if the response sound 12 received by the in-ear sound receiver 20 is higher than the response sound threshold corresponding to the user 90's chewing behavior pre-stored in the response sound threshold database 51, it is then determined that the currently received speech signal 61 is the sound of the user 90 chewing food, and therefore the sound processing unit 30 will lower the output volume 41 outputted from the speaker 40. If the sound processing unit 30 determines that the response sound 12 is lower than the response sound threshold corresponding to the user behavior mode, it means that the currently received speech 61 does not come from the user 90. At this time, the sound processing unit 30 will not adjust the output volume 41 outputted from the speaker 40 (step S5) so that the user 90 may hear the speech signal 61 clearly.

Please note that, according to one embodiment of the present invention, the response sound threshold database 51 can store a plurality of response sound thresholds each respectively corresponding to a user behavior mode. For example, the user behavior modes such as the user's own speaking, chewing and swallowing respectively have their corresponding response sound thresholds. Therefore, whenever the sound processing unit 30 determines that the response sound 12 is higher than the response sound threshold of any user behavior mode, the sound processing unit 30 will then lower the output volume 41 outputted from the speaker 40.

Furthermore, because the shape of each individual's ear canal will be correspondingly deformed by different individual behaviors such as eating and speaking, and the frequency responses of the high frequency test sound 11 reflected by ear canals of different shapes are subject to different behavior modes of different persons, it is required that the user 90 establish a corresponding response sound threshold database 51 prior to the first use of the hearing assistance device 1 of the present invention. In this embodiment, when the user 90 follows instructions from the hearing assistance device 1 to take different actions, the in-ear speaker 10 will play a test audio signal within a frequency range, and the in-ear sound receiver 20 will receive sounds accordingly, in order to analyze dynamic frequency response modes of the user's ear canal 91 in different behavior states (such as speaking, chewing or swallowing), and to utilize the data for calculating the response sound thresholds each respectively corresponding to one of the different user behavior modes, so that the response sound thresholds can be later used as a comparison reference specifically for the user 90 who wears the hearing assistance device 1.

According to the abovementioned description, the hearing assistance device 1 and the method of adjusting an output sound of the hearing assistance device of the present invention are capable of adapting to deformation of the shape of an ear canal caused by human facial movements (for example mandibular movements), utilizing the in-ear speaker 10 for emitting a high frequency test sound 11 into the user's ear canal 91, utilizing the in-ear sound receiver 20 of the hearing assistance device 1 for receiving the response sound 12 generated after the high frequency test sound 11 is reflected by the user's ear canal 91, determining a user behavior mode, and thereby identifying whether the speech signal 61 received by the hearing assistance device 1 is the user's own voice. If yes, the hearing assistance device 1 will lower the volume; otherwise, the volume will not be adjusted. As a result, the present invention achieves the object of lowering the volume of a sound from the user wearing the hearing assistance device 1, thereby eliminating the problem of unconditional amplification of all received sounds existing in conventional techniques.

Although the present invention has been explained in relation to its preferred embodiments, it is to be understood that many other possible modifications and variations can be made without departing from the spirit and scope of the invention as hereinafter claimed.

What is claimed is:

1. A hearing assistance device, worn in an ear of a user, the ear comprising a user's ear canal, and the hearing assistance device comprising:
a speaker for outputting a speech signal;
an in-ear speaker for emitting a high frequency test sound, wherein the frequency of the high frequency test sound is higher than 15 kHz and lower than 30 kHz;
an in-ear sound receiver for receiving a response sound after the in-ear speaker emits the high frequency test sound; and
a sound processing unit for determining whether the response sound is higher than a response sound threshold; and, if yes, adjusting an Output volume of the speech signal.

2. The hearing assistance device as claimed in claim 1, further comprising a response sound threshold database for storing response sound thresholds respectively corresponding to user behavior modes.

3. The hearing assistance device as claimed in claim 1, wherein the sound processing unit lowers the output volume of the speech signal according to a user behavior mode.

4. The hearing assistance device as claimed in claim 1, wherein if the sound processing unit determines that the response sound is lower than the response sound threshold corresponding to a user behavior mode, the sound processing unit will not adjust the output volume of the speech signal.

5. The hearing assistance device as claimed in claim 1, wherein the sound processing unit determines whether the response sound is higher than the response sound threshold corresponding to a user behavior mode; and, if yes, the sound processing unit adjusts the output volume of the speech signal.

6. The hearing assistance device as claimed in claim 5, wherein the user behavior mode comprises a plurality of user behavior modes each respectively corresponding to one of the response sound thresholds, and the response sound thresholds are stored in a response sound threshold database.

7. A method of adjusting an output sound of a hearing assistance device, comprising:
emitting a high frequency test sound, wherein the frequency of the high frequency test sound is higher than 15 kHz and lower than 30 kHz;
receiving a response sound after the high frequency test sound is emitted;
determining whether the response sound is higher than a response sound threshold; and
if yes, adjusting an Output volume.

8. The method as claimed in claim 7, wherein the hearing assistance device comprises a response sound threshold database for storing response sound thresholds respectively corresponding to user behavior modes.

9. The method as claimed in claim 7, wherein adjusting an output volume means lowering the output volume.

10. The method as claimed in claim 7, wherein if the response sound is lower than the response sound threshold corresponding to a user behavior mode, the sound processing unit will not adjust the output volume of a speech signal.

11. The method as claimed in claim 7, further comprising a step of determining whether the response sound is higher than the response sound threshold corresponding to a user behavior mode; and, if yes, adjusting the output volume.

12. The method as claimed in claim 11, wherein the user behavior mode comprises a plurality of user behavior modes each respectively corresponding to one of the response sound thresholds, and the response sound thresholds are stored in a response sound threshold database.
